(19) United States
(12) Patent Application Publication
(10) Pub. No.: US 2009/0191137 A1
(43) Pub. Date: Jul. 30, 2009

(54) Method and Material for Controlling or Eliminating Potentially Harmful, Contaminating or Nuisance Micro-Organisms or Cells

(76) Inventors: Rajan K. Vempati, Plano, TX (US); Richard E. Wagner, Bloomington, IN (US)

Correspondence Address: WHITAKER, CHALK, SWINDLE & SAWYER, LLP, 3500 CITY CENTER TOWER II, 301 COMMERCE STREET, FORT WORTH, TX, 76102-4186, US

(21) Appl. No.: 12/257,936

(22) Filed: Oct. 24, 2008

Related U.S. Application Data
(60) Provisional application No. 60/982,451, filed on Oct. 25, 2007.

(51) Int. Cl. A61K 8/19; C02F 1/50; A61L 2/16; A61K 33/32; A61Q 11/00

(52) U.S. Cl. 424/49; 210/764; 422/28; 424/639

(57) ABSTRACT

A method is shown for reducing or eliminating the levels or activities of potentially harmful or contaminating organisms or cells by applying nanophase manganese (VII) oxide to solutions, surfaces or materials to eliminate, reduce or prevent the growth of potentially harmful, contaminating or undesirable microorganisms, such as algae and bacteria.

### CROSS REFERENCE TO RELATED APPLICATIONS

The present invention claims priority from a provisional application Ser. No. 60/982,451, filed Oct. 25, 2007, entitled “Method And Material For Controlling Or Eliminating Potentially Harmful, Contaminating or Nuisance Micro-organisms”, by the same inventors.

### BACKGROUND OF THE INVENTION

1. Field of the Invention

This invention relates to methods and materials for reducing or eliminating the levels or activities of potentially harmful or contaminating organisms or cells. More particularly, the invention relates to reducing or eliminating the level or activity of potentially harmful organisms or cells by applying nanophase manganese oxide (VII) to solutions, surfaces or materials to eliminate, reduce or prevent the growth of potentially harmful, contaminating or undesirable microorganisms, such as algae and bacteria.

2. Description of the Prior Art

There are vast numbers of organisms and cells that are harmful to humans, animals and plants. Many microorganisms infect humans, animals and plants causing diseases. These diseases can be life threatening—such as tuberculosis—or be more subtle nuisances such as skin diseases (athlete's foot). Microorganisms can also cause damage to familiar products and structures, such as clothing, shingles and wood. Control of harmful microorganisms and cells has often focused on the preventative or curative application of chemicals designed to reduce or eliminate these microorganisms or cells. Broadly, these are referred to as biocides and biostatics.

These biocidal or biostatic chemicals can range from specific chemicals with specific modes of action that act on specific sites—such as antibiotics which bind to a the target microorganism's ribosome—to broad spectrum chemicals—such as sodium hypochlorite (bleach)—that act on a number of cellular functions including protein and membrane structure. Examples of preventative chemical treatments include treatment of water supplies with ozone, filters to eliminate pathogens from the air, pre- and post-harvest sprays to prevent mold on fruit and vegetables, and rinses to reduce plaque-forming bacteria in the oral cavity. Examples of curative treatments include washing contaminated hospital bedding, horticultural instruments and vinyl-siding with bleach, as well as treating systemic infection with antibiotics. While the mode of action and structure of these chemicals is diverse, their intended use is the same: to reduce or eliminate the level or activity of harmful microorganisms and cells.

Effectiveness of these biocidal and biostatic chemicals can vary depending upon the dose, formulation, concentration, timing of application, species of microorganism, type of cell, age and stage of development of the microorganism or cell, environment and medium (surface, liquid, gas) to which they are applied.

In general there is concern about the ability of microorganisms to overcome the biocidal or biostatic effect of the chemical. In general, resistance in microorganisms and cells is higher for biostatic or biocidal chemicals that have more specific targets and modes of action. A common example would be that of antibiotic resistance. It is more difficult for microorganisms to develop resistance to broad spectrum chemicals with nonspecific targets or modes of action. Examples of these in addition to those aforementioned include hydrogen peroxide and copper sulfate.

### SUMMARY OF THE INVENTION

This patent describes a method for reducing or eliminating the level or activity of potentially harmful microorganisms or cells with a biocidal/biostatic agent based upon a special form of manganese oxide. A particularly preferred manganese oxide material is a nanophase manganese oxide stabilized in the (VII) oxidation state, hereinafter referred to as NM7O, an abbreviation for nanophase Mn (VII) oxide. The NM70 is preferably supplied on a solid support such as a clay or zeolite.

The insoluble NM7O is applied to water to eliminate or prevent the growth of potentially harmful or contaminating microorganisms. Exemplary harmful organisms include algae, bacteria and viruses. The water can be contained in a small vial or a large lake. The liquid can be used for pharmaceutical purposes such as a carrier or diluent for a drug; for recreational purposes such as swimming pools or lakes; for drinking water such as reservoirs or point sources (taps); for cooling purposes such as those application in cooling towers for power plants, buildings or homes; for municipal, industrial and agricultural waste including sewage; or for aquaculture. The NM70 can be applied before, during or after presence of the potentially harmful or contaminating microorganisms is detected. Therefore used as a preventive and/or proactive prophylactic agent. In another embodiment, the NM7O is applied to the surface of a material to eliminate or prevent the growth of potentially harmful or contaminating microorganisms. Exemplary surfaces include fabrics used for tarps, bedding or military purposes. Other examples include surfaces used for horticultural purposes and meat processing purposes.

In another embodiment, the NM70 is added to a material prior to its fabrication into a material or cloth to eliminate or prevent the growth of potentially harmful or contaminating microorganisms. Exemplary materials include cotton, wool, non-wovens and melt blown fabrics, polyester, paper and rayon. The materials can be used for making clothing, filters, coverings or bedding.

In still another embodiment the NM70 is added to a paste or other carrier, e.g., a mouth rinse, for delivery to the oral cavity for control of to eliminate or prevent the growth of potentially harmful or contaminating microorganisms. Exemplary microorganisms include Streptococcus mutans and S. sobrinus, Lactobacillus acidophilus, Actinobacillus actinomycetemcomitans, Porphyromonas gingivalis, Prevotella intermedia, Bacteroides forsythus, Fusobacterium species, Campylobacter rectus, and Treponema denticola. Thus, there are a number of microorganisms that may be targeted by this technology.

In still another embodiment, the NM7O is added to a feed or food product to eliminate or prevent the growth of potentially harmful or contaminating microorganisms. In this embodiment the NM7O can be used as a preservative to prevent the growth of potentially harmful or contaminating microorganisms or as a medicant to prevent the growth of potentially harmful or contaminating microorganisms inside the animal. Exemplary feeds include aquafeeds and feeds for companion animals. Exemplary organisms controlled include bacteria in the shrimp gut.

The previous embodiments are provided as examples and do not represent the entirety of all applications of NM7O and other manganese oxidative states for eliminating or preventing the growth of potentially harmful, nuisance or contaminating microorganisms or cells.

Additional objects, features and advantages will be apparent in the written description which follows.

### BRIEF DESCRIPTION OF THE DRAWINGS

FIG. 1 is a comparison of untreated and NM7O treated algal suspensions, the treated suspension being shown on the right.

FIG. 2 illustrates the re-growth of Chlamydomonas from flasks of treated algae that appeared cleared following treatment with lower levels (<3 ppm) of NM7O. The plate on the left shows algal growth from the control, whereas the plate on the right shows re-growth of individual colonies from cultures treated with lower concentrations of NM7O.

FIG. 3 is a comparison of reaction with algal cells treated with NM7O showing, from left to right, the control (first tube), NM7O treated (second tube) and copper sulfate treated (third tube).

FIG. 4 shows on the left an E. Coli Petri dish with 1×E8 cells and, on the right, an NM7O treated sample with four colonies.

FIG. 5 illustrates the growth of algae in fabrics untreated in the two beakers on the left and treated with NM7O in the two beakers on the right.

FIG. 6 shows a control cotton sample, followed by washed cotton with NM7O, unwashed cotton with NM7O, cotton treated with Mn(III) oxide, and polyurethane treated with Mn(VIII) oxide.

### DETAILED DESCRIPTION OF THE INVENTION

The novel nanophase Mn(VII) oxide (referred to herein as “NM7O”) which is used in the practice of the invention will first be described. This material is a stable and strong Lewis acid and can be stabilized in both the supported (for example on a zeolite or clay) and unsupported forms. The NM7O can be synthesized by reacting 1,4 phenylenediamine compound with Mn(II) mineral at a specified pH. This is a single step, simple and rapid process. NM7O has the capacity to destroy Lewis bases, e.g., N, S, O and P containing lone pairs of electrons and is effective in both polar and non-polar solvents, the redox reaction occurring on its surfaces, with no Mn being released into the solvent.

A detailed description of NM7O, as well as its method of manufacture and characterization data, are provided in U.S. Pat. No. 6,953,763, entitled “Solid Support Stabilized Mn(III) and Mn(VII) And Method of Preparation”, issued Oct. 11, 2005, to Vempati and Son, the entire disclosure of which is incorporated herein by reference. The following example describes the basic preparation of the NM7O which is used in the practice of the present invention:

### Protocol:

NM7O was synthesized by adding 1,4-phenylenediamine to Mn(II) mineral and/or Gonzalez clays. The Mn(VII) oxidation state was determined by cyclic voltammetry and optical spectroscopy in the visible region.

### Mn(VII) Oxide (NM70) Synthesis:

The solid support material used was a hydrophilic bentonite clay. To a-250-mL glass beaker containing a magnetic bar, 18 g of MnCl₂ was dissolved in 100 mL of distilled water and placed on a magnetic stirrer. After 15 min, 50 g of bentonite clay was added and the suspension equilibrated for 15 min. Then, the pH raised to 8.5 using NaOH, resulting in beige colored precipitation of Mn(II) mineral on the clay surfaces. After 30 min of equilibration, 1 g of 1,4-PDA was added and temperature of the beaker raised to 70° C.; following three hrs of stirring the suspension color changed to violet indicating the formation of nanophase Mn(VII) oxide. The material was either stored as slurry or air-dried at 80° C. The clay contained a 10% Mn coating.

Further examples of the preparation of various Mn oxides, as well as characterization data for the NM7O material which is the subject of the present invention can be found in the previously referenced U.S. Pat. No. 6,953,763. The present invention deals with additional novel uses of the previously described NM7O which were not appreciated at the time of the initial work which was done in synthesizing NM7O.

The use of the previously described NM7O for the purposes of the invention will now be described. The following examples are used for the purpose of illustration only and are not intended to limit the scope of the invention as defined in the claims which are appended hereto. The NM70 used in the examples was described in U.S. Pat. No. 6,953,763 on clay support.

### Example 1

### Application of NM70 for Elimination or Preventing the Growth of Algae

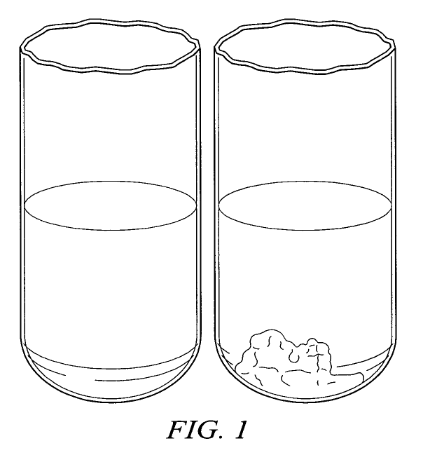

Studies were conducted that demonstrate the potential of NM7O as an algaecide. When added to a suspension of algae, the product rapidly clears the solution of the algae (see FIG. 1). Note the clarity of the solution and accumulation of killed algae at the bottom of the tube on the right. Upon reaction with the algae, both product and algae drop to the bottom of the tube. In addition, the product changes from violet to brown, indicating a change from the nanophase Mn(VII) oxidation state to Mn(IV) oxidation state. The color change can serve as a valuable indicator of activity, as untreated NM7O remains suspended and violet colored.

Preliminary experiments have indicated that the product is active within a range of 1 to 10 ppm; this corresponds to an active ingredient of 0.3-1 ppm. Experiments with different concentrations of NM7O and algae suggest that a linear relationship exists between the amount of NM7O added and the amount of algae killed.

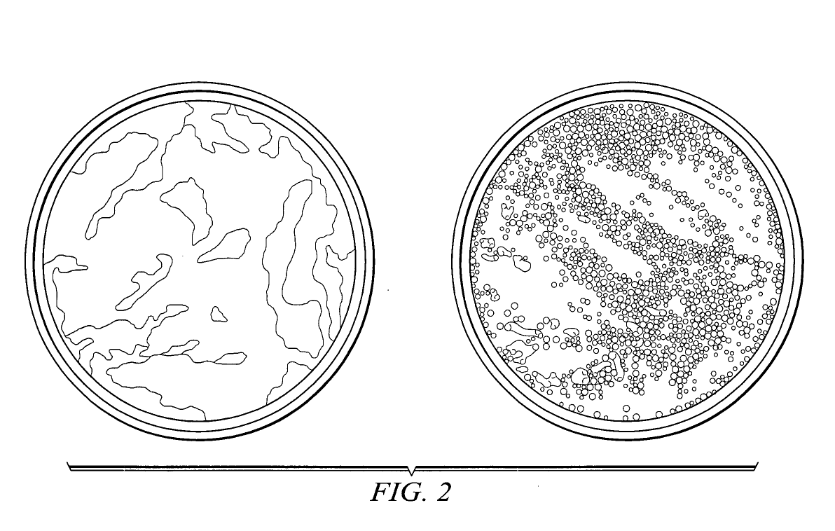

When levels of algae were higher then a certain threshold relative to a fixed amount of NM7O, re-growth occurred. This may be due to the requirement of direct contact between the product and the algae. Re-growth is not uncommon following treatment with herbicides and is species and concentration dependent, with some products being algaecidal and some products being algae static (FIG. 2).

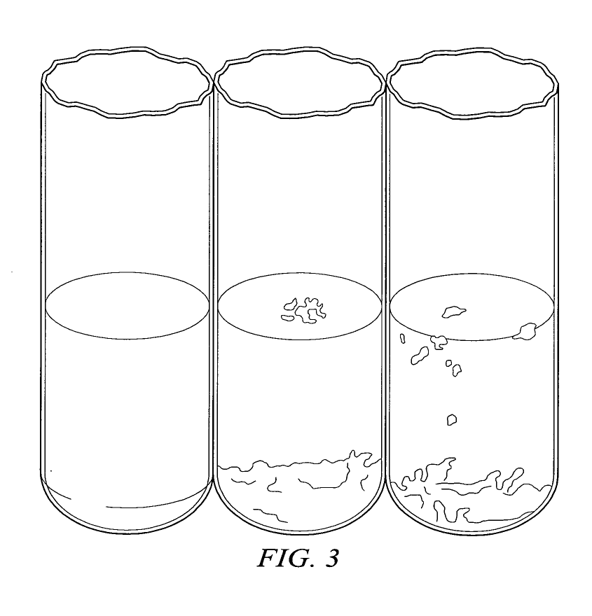

Comparisons were made with copper sulfate added to separate tubes containing the same concentration of algae (1×10⁶ cells/ml) as the experiments conducted with NM7O (FIG. 3). As was demonstrated with respect to the NM7O in FIG. 1, the algae clumped and sunk to the to the bottom of the tube; however, in contrast to the clearing effect of NM7O, the solution turned blue, due to the cupric ion. Microscopic evaluation of the sediment revealed that the cells treated with NM7O appeared as hollow shells suggesting that the plasma membrane had been destroyed leaving only the cell wall, which has a high protein content. In contrast the cells treated with copper sulfate appeared intact with full cellular contents. From this observation, it appears that the mechanisms of the two algaecides are different. NM7O may behave more like hydrogen peroxide, another potent oxidizing agent, which destroys membranes by a free radical mechanism. Interestingly, NM7O is a stronger oxidizing agent than hydrogen peroxide: when the two compounds (peroxide >30%) are combined the reaction is violent and color change spontaneously from violet to brown, indicating a change from the Mn(VII) oxidation state to Mn (IV) oxidation state. The H₂O₂ is decomposed to O₂ and H₂O vapor.

### Example 2

### Application of NM70 for Eliminating or Preventing the Growth of Bacteria

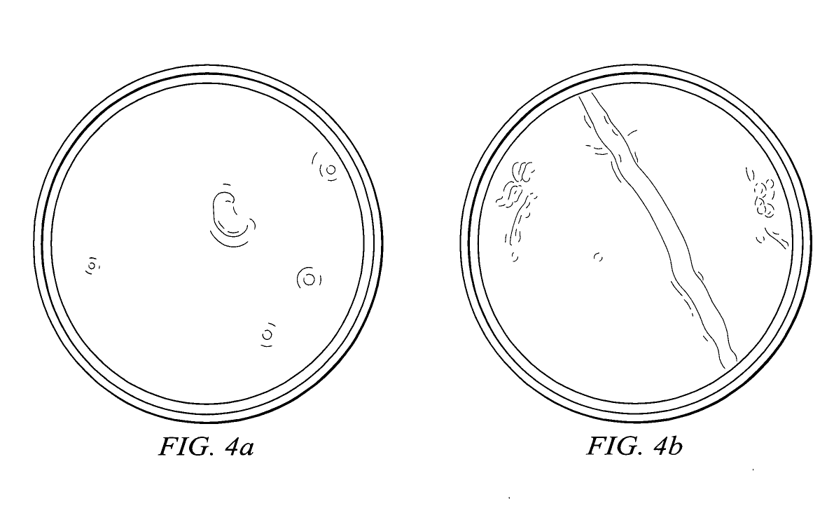

Several test tubes containing LB media were inoculated with a single colony of E. coli and allowed to grow for 14 hrs at 37° C. Bacterial suspensions were treated or not treated with NM7O. Tubes were returned to the incubator for another 14 hrs. One tube was kept untreated as a control. The tubes were then plated on LB agar with 100 μl of sample from the tubes (see FIGS. 4a and 4b). The Petri plates were returned to the incubator overnight. As can be seen from FIGS. 4a and 4b, the control sample grew as expected and produced a lawn on the LB media plate (on the left—note the strip of bacteria removed (arrow) using a transfer loop). The tubes that were treated with the NM70 showed significant inhibition of growth (see FIG. 4b). The plate yielded only four colonies. Assuming conservatively that the cells were in late log phase and there would be approximately 1×10⁸ cells, if all cells survived—as in the control—one would expect 1×10⁶ cells to be delivered to the plate (100 μl sample), resulting in the observed lawn. Since only 4 cells (see arrows) grew the survival rate was 4/1×10⁶ or 0.0004%.

### Example 3

### Application of NM70 to Fabrics for Eliminating or Preventing the Growth of Algae

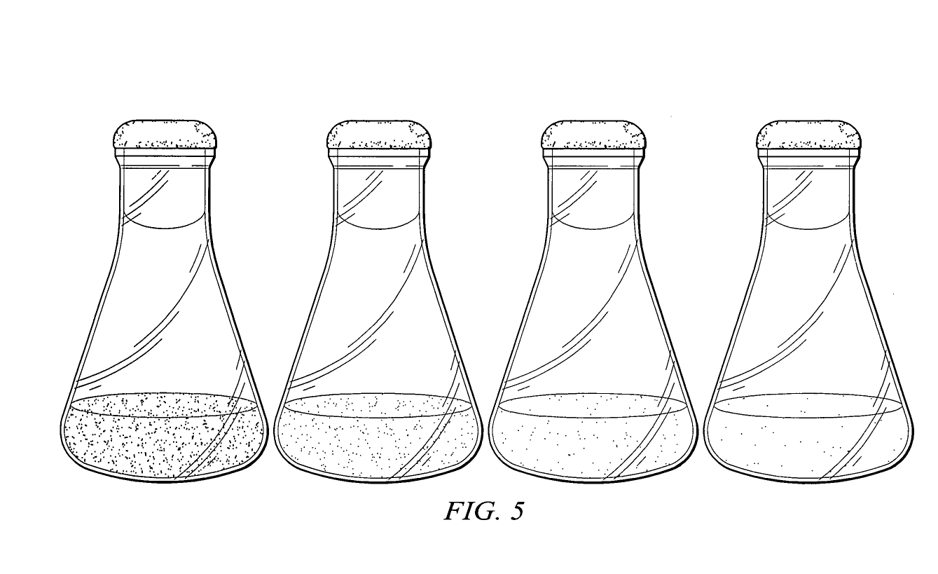

Treatment of fabrics coated with nanophase Mn(VII) oxide with a suspension of algae demonstrated that the fabrics had excellent algaecide activity (see FIG. 5). Cotton fabrics treated with Mn(VII) oxide were cut into approximately 0.5 in×0.5 in squares. The samples were inoculated with 200 μl (3×10E6 cells/ml) of a three day culture of Chlamydomonas reinhardtii (green algae). The samples were kept in separate Petri dishes and sealed with parafilm. The dishes were kept at 20-25° C. for 24 hrs. The individual fabric samples were then placed into 125 ml Erlenmeyer flask containing 50 mls of TAP media. The flasks were placed on a platform shaker for 3 days in full light and at 20-25° C. with the shaker set at 130 rpm. As seen in FIG. 5, the two flasks on the left containing the cotton not treated with nanophase Mn(VII) oxide supported vigorous algal growth. The two flasks on the right containing the nanophase Mn(VII) oxide treated cotton showed no sign of algal growth indicating algaecidal activity of the NM70.

### MnVII Treated Materials

The following materials were supplied to be tested for their algaecidal abilities: Untreated Cotton, Washed Cotton treated with NM7O, Cotton treated with NM7O, Cotton treated with NM3O, and Polyurethane treated with NM7O. Samples of the supplied materials were cut into approximately 0.5 in×0.5 in squares. The samples were inoculated with 200 μl (3×10E6 cells/ml) of a-three day culture of Chlamydomonas reinhardtii (green algae). The samples were kept in separate Petri dishes and Para filmed. The dishes were kept at 20-25° C. for 24 hrs. The individual samples were then placed into 125 ml Erlenmeyer flask containing 50 mls of TAP media. The flasks were placed on a platform shaker for three days in full light and at 20-25° C. with the shaker set at 130 rpm.

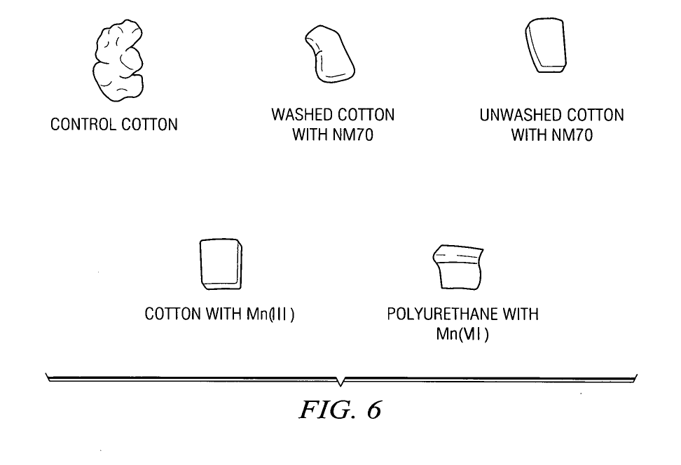

As seen in FIG. 6, the control cotton turned green with algae. The washed cotton treated with NM7O also grew but it was far less green than the control. All other samples showed no signs of algal growth.

### FURTHER USES OF THE TECHNIQUES OF THE INVENTION

As will be apparent from the foregoing, the method of the invention provides a convenient technique for controlling or preventing the growth of microorganisms in an aqueous solution by merely adding nanophase manganese (VII) oxide to the aqueous solution. The aqueous solution can then be used to control or prevent the growth of microorganisms such as algae and bacteria. Example microorganisms might also include viruses, fungi, mycoplasma, helminthes and living cells. In some cases, the microorganism may be a parasite or pathogen.

The previously described nanophase manganese (VII) oxide may be attached to a solid support prior to delivery to the water. Preferred solid support materials are described in detail in the previously referenced U.S. Pat. No. 6,953,763, entitled “Solid Support Stabilized Mn(III) and Mn(VII) And Method of Preparation”, the disclosure of which has been incorporated herein by reference.

The previously described “solution” to which the nanophase manganese(VII) oxide is added may include such things as an aquarium, an aquafarm, a pond, a lake, a swimming pool, drinking water or effluent. The effluent may be from a municipal, agricultural or industrial source, including cooling towers, settling ponds, and the like.

Preferably, the nanophase manganese (VII) oxide is added prior to detectable levels of the microorganism developing. The step of preventing the growth of microorganisms in an aqueous solution growth may be accomplished by killing a cell of the microorganism. Microorganisms of the type under consideration include plants, algae, bacteria, viruses, fungi, mycoplasma and helminthes.

The microorganism may also be a parasite or pathogen.

The aqueous solution may also contain a pharmacological agent in addition to the nanophase manganese (VII) oxide. In some cases, the pharmacologic agent may be a vaccine. The pharmacological agent may also be physiological saline.

The method of the invention envisions the step of adding nanophase manganese (VII) oxide to a suitable support surface prior to contact with the microorganism. This method of delivery can be used, for example, for manufacturing hospital beds, diapers, medical gauzes, biochemical warfare suites, mats for sterilization rooms, clothing for health workers, hazmat suits, mold resistant particle board, as well as incorporated into liquids including paints, polymers, latex, organic and inorganic solvents, etc. Preferred solid support materials thus include fabrics, clays, zeolites and lime mixtures.

The surface to which the nanophase manganese (VII) oxide is attached may be a fiber. The fiber may then be used for making a fabric. The fabric, in turn, may be used in making cloths, tarps, coverings, ropes and filters.

The treatment target of the nanophase manganese (VII) oxide materials of the invention may be a part of a living organism, such as a tooth. The nanophase manganese (VII) oxide can be mixed with a toothpaste prior to the delivery to the surface. The NM70 can also be included in a mouth rinse solution, or mouth wash.

In similar fashion, the NM7O may be added to a feed or food product to eliminate or prevent the growth of potentially harmful or contaminating microorganisms. Example feeds include aquafeeds and feeds for companion animals. One example of the control or an undesirable microorganism in such as circumstance would be the control of bacteria in the shrimp digestive track.

An invention has been provided with several advantages. The method of the invention provides a technique for reducing or eliminating the level or activity of potentially harmful organisms or cells through the use of nanophase manganese (VII) oxide. The NM7O can be conveniently applied to solutions, surfaces or materials to eliminate, reduce or prevent the growth of potentially harmful, contaminating or undesirable microorganisms, such as algae and bacteria.

While the invention has been described in several of its forms, it is not thus limited, but is susceptible to various changes and modifications without departing from the spirit thereof.

We claim:

1. A method for controlling or preventing the growth of microorganisms in an aqueous solution comprising adding nanophase manganese (VII) oxide to the aqueous solution.

2. The method of claim 1, wherein the microorganisms are algae.

3. The method of claim 1, wherein the microorganisms are bacteria.

4. The method of claim 1, wherein the microorganisms are selected from group consisting of viruses, fungi, mycoplasma, helminthes and living cells.

5. The method of claim 1 wherein the microorganism is a parasite or pathogen.

6. The method of claim 1 wherein the nanophase manganese (VII) oxide is attached to a solid support prior to delivery to the water.

7. The method of claim 1, wherein the aqueous solution is selected from the group consisting of an aquarium, an aquafarm, a pond, a lake, a swimming pool, drinking water and effluent.

8. The method of claim 7, wherein the effluent is from a municipal, agricultural or industrial source, including cooling towers, settling ponds, and the like.

9. The method of claim 1, wherein the nanophase manganese (VII) oxide is added prior to detectable levels of the microorganism developing.

10. The method of claim 1, wherein the step of preventing the growth of microorganisms in an aqueous solution growth is accomplished by killing a cell of the microorganism.

11. An aqueous solution for controlling or preventing the growth of microorganisms, said solution comprising nanophase manganese (VII) oxide and water.

12. The aqueous solution of claim 11, wherein the solution contains a pharmacological agent in addition to the nanophase manganese (VII) oxide.

13. The aqueous solution of claim 12, wherein the pharmacologic agent is a vaccine.

14. The aqueous solution of claim 13, wherein the pharmacological agent is a physiological saline.

15. A method for controlling or preventing the growth of microorganisms on a surface, the method comprising the steps of adding nanophase manganese (VII) oxide to the surface of a solid support, the solid support being thereafter exposed to the microorganism.

16. The method of claim 15, wherein the solid support is selected from the group consisting of fabrics, clays, zeolites and lime mixtures.

17. The method of claim 15, wherein the microorganisms are selected from the group consisting of plants, algae, bacteria, viruses, fungi, mycoplasma and helminthes.

18. The method of claim 15, wherein the microorganism is a parasite or pathogen.

19. The method of claim 15, wherein the surface is a fiber.

20. The method of claim 19, wherein the fiber is used for making a fabric.

21. The method of claim 20, wherein the fabric is used making cloths, tarps, coverings, ropes and filters.

22. The method of claim 15, wherein surface is a tooth.

23. The method of claim 22, wherein the nanophase manganese (VII) oxide is mixed with a toothpaste prior to the delivery to the surface.
